(19) United States
(12) Patent Application Publication
(10) Pub. No.: US 2025/0352602 A1
(43) Pub. Date: Nov. 20, 2025

(54) DIETARY SUPPLEMENTS AND METHODS FOR REDUCING INFLAMMATION

(71) Applicant: Melaleuca, Inc., Idaho Falls, ID (US)

(72) Inventors: Jordan Joy, Idaho Falls, ID (US); Nasser A. Fredj, Idaho Falls, ID (US)

(21) Appl. No.: 19/205,001

(22) Filed: May 12, 2025

Related U.S. Application Data
(60) Provisional application No. 63/647,968, filed on May 15, 2024.

(51) Int. Cl. A61K 36/82; A61K 31/09; A61K 31/353; A61K 31/7048; A61K 36/9066; A61K 36/9068; A61P 29/00

(52) U.S. Cl. CPC A61K 36/82; A61K 31/09; A61K 31/353; A61K 31/7048; A61K 36/9066; A61K 36/9068; A61P 29/00

(57) ABSTRACT

Dietary supplement compositions containing bioavailability-enhanced curcuminoids are provided herein, as are methods and materials for reducing inflammation and stiffness associated with normal physical activity and exercise, as well as inflammation and stiffness associated with conditions such as arthritis and osteoarthritis.

### CROSS-REFERENCE TO RELATED APPLICATIONS

This application claims benefit of priority from U.S. Provisional Application Ser. No. 63/647,968, filed May 15, 2024. The disclosure of the prior application is considered part of (and is incorporated by reference in) the disclosure of this application.

### TECHNICAL FIELD

This document relates to methods and materials for reducing pain, inflammation, and stiffness associated with inflammatory conditions. For example, this document relates to dietary supplements and methods of their use to reduce inflammation and/or pain associated with physical activity or conditions such as arthritis and osteoarthritis.

### BACKGROUND

Inflammatory conditions such as arthritis and osteoarthritis (OA) are serious medical problems that affect many people throughout the world. For example, arthritis is one of the most prevalent chronic health problems in the United States, with an estimated 43 million Americans suffering from some form of arthritis. Treatments that can relieve pain, inflammation, and discomfort associated with OA often involves the use of non-steroidal anti-inflammatory drugs (NSAIDs). NSAIDs also can be used to treat pain and inflammation associated with exercise or normal physical activity. However, NSAIDs can cause adverse effects such as gastric bleeding, liver damage, and kidney damage. In addition, long-term use of NSAIDs can lead to reduced effectiveness. Thus, there is a need for improved treatment regimens for patients suffering from inflammatory conditions.

### SUMMARY

This document provides methods and materials related to reducing pain, inflammation, and/or stiffness associated with physical activity (e.g., exercise-induced inflammation or joint pain) or associated with inflammatory conditions such as arthritis and OA. For example, this document provides compositions (e.g., dietary supplements) containing one or more bioavailability-enhanced curcuminoids (e.g., bioavailability enhanced turmeric containing one or more curcuminoids), an iridoid (e.g., an iridoid glycoside), a green tea extract, and a ginger component. In some cases, a composition provided herein can contain a bioavailability-enhanced curcuminoid, a Devil's Claw extract, a green tea extract, and/or a ginger component.

In a first aspect, this document features a dietary supplement. The dietary supplement can contain, consist essentially of, or consist of one or more bioavailability-enhanced curcuminoids, one or more iridoids, green tea extract, and one or more ginger components. The one or more bioavailability-enhanced curcuminoids can include a component of a turmeric extract (e.g., bioavailability enhanced turmeric). The one or more iridoids can be selected from the group consisting of: harpagoside, loganin, sweroside, vogeloside, and epi-vogeloside. The one or more iridoids can include a component of a plant extract (e.g., a Devil's Claw extract). The green tea extract can contain one or more of catechin, epicatechin, gallocatechin, epigallocatechin, epicatechin gallate, epicatechin gallate, and epigallocatechin gallate. The green tea extract can contain epigallocatechin gallate. The one or more ginger components can include a ginger root extract. The dietary supplement can be in the form of a tablet, a capsule, a powder, or a liquid.

In another aspect, this document features a dietary supplement that contains, consists essentially of, or consists of (a) from about 1 to about 150 mg of bioavailability-enhanced curcuminoid, (b) from about 40 to about 60 mg of Devil's Claw extract, (c) from about 150 to about 350 mg of green tea extract, and (d) from about 40 to about 60 mg of ginger component. The dietary supplement can contain, consist essentially of, or consist of (a) about 50 mg/serving of the bioavailability-enhanced curcuminoid, (b) about 52.5 mg/serving of the Devil's Claw extract, (c) about 250 mg/serving of the green tea extract, and (d) about 52.5 mg/serving of the ginger component.

In another aspect, this document features a dietary supplement that contains, consists essentially of, or consists of (a) from about 1 to about 150 mg of bioavailability-enhanced curcuminoid, (b) from about 40 to about 60 mg of Devil's Claw extract, (c) from about 150 to about 350 mg of green tea extract, and (d) from about 40 to about 60 mg of ginger component. The dietary supplement can contain, consist essentially of, or consist of (a) about 50 mg/serving of the bioavailability-enhanced curcuminoid, (b) about 52.5 mg/serving of the Devil's Claw extract, (c) about 250 mg/serving of the green tea extract, and (d) about 52.5 mg/serving of the ginger component.

In another aspect, this document features a method for promoting a healthy inflammatory response to normal daily activity in a mammal. The method can include, consist essentially of, or consist of administering to the mammal a dietary supplement provided herein. The method can include administering the dietary supplement to the mammal on a daily basis for at least one week. The administering can be effective to reduce inflammation in the mammal. The mammal can be a human.

In another aspect, this document features a method for promoting a healthy inflammatory response to exercise-induced inflammation in a mammal. The method can include, consist essentially of, or consist of administering to the mammal a dietary supplement provided herein. The method can include administering the dietary supplement to the mammal on a daily basis for at least one week. The administering can be effective to reduce inflammation in the mammal. The mammal can be a human.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a bar graph plotting the results of a human ex vivo experiment to assess the free-radical quenching capacity of the Test Formula and a Benchmark product. Human participants submitted an aliquot of capillary blood that was spiked with the proinflammatory cytokine, TNF-α, and the free radical quenching capacity was measured by electron paramagnetic resonance immediately prior to and 2 hours after supplementing with one serving of Test Formula or Benchmark in a randomized, within-subject, crossover design.

FIG. 2 is a bar graph plotting the results of a 12-week study in a self-reported healthy human population that did not have high baseline inflammation or high joint pain (based on WOMAC scores) to compare the levels of TNF-α spiked reactive oxygen species (ROS), ROS, high-sensitivity C-reactive protein (hsCRP), Western Ontario and McMaster Universities Arthritis Index (WOMAC) score, WOMAC pain, WOMAC stiffness, and WOMAC physical function when supplemented with either control or Test Formula.

### DETAILED DESCRIPTION

This document provides methods and materials for reducing pain, inflammation, and/or stiffness associated with inflammatory conditions such as, without limitation, arthritis and OA in a mammal (e.g., a human, a non-human primate, a rodent, a dog, a pig, or a rabbit). For example, the document provides compositions (e.g., dietary supplements) containing, or consisting essentially of, a bioavailability-enhanced curcuminoid, an iridoid (e.g., an iridoid glycoside), a green tea extract, and a ginger component. In some cases, a composition provided herein can contain a bioavailability-enhanced curcuminoid, a Devil's Claw extract, a green tea extract, and/or a ginger component.

### Bioavailability-Enhanced Turmeric/Curcuminoid

The compositions provided herein can contain one or more bioavailability-enhanced curcuminoids (e.g., bioavailability enhanced turmeric containing one or more curcuminoids). Curcuminoids are polyphenols that can be obtained from turmeric rhizomes (Curcuma longa). Non-limiting examples of curcuminoids include curcumin, desmethoxycurcumin, and bisdemethoxycurcumin. Curcuminoids can have beneficial antioxidant, anti-inflammatory, anticancer, and immunomodulatory effects on mammals, but their effectiveness is limited by low water solubility and inadequate bioavailability, particularly at pH above 7. See, e.g., Gupta et al., Clin. Exp. Pharmacol. Physiol., 39:283-299, 2012; Giordano and Tommonaro, Curcumin and Cancer. Nutrients, 11:2376, 2019; Yabas et al., Front. Immunol., 12:157, 2021; and Liu et al., J. Drug Target., 24:694-702, 2016.

Bioavailability-enhanced curcuminoids can have improved water solubility, and their intestinal absorption can be facilitated by protection from degradation at alkaline intestinal pH conditions (see, e.g., Yabas et al., supra). Bioavailability-enhanced curcuminoids can be synthesized or can be derived from natural sources. In some cases, a bioavailability-enhanced curcuminoid can be a component of a plant extract. For example, a bioavailability-enhanced curcuminoid can be a component of an extract of turmeric. An extract of turmeric can be prepared using, for example, ethanol or hydroalcoholic extraction of turmeric. In some cases, bioavailability-enhanced curcuminoids or plant extracts containing bioavailability-enhanced curcuminoids (e.g., turmeric extracts) can be obtained commercially. For example, a bioavailability-enhanced curcuminoid known as Next Generation Ultrasol Curcumin (commercialized as CURCUWIN® Ultra+ or CU+) can be obtained from OmniActive Health Technologies (Mumbai, India). Other bioavailability-enhanced curcuminoid formulations include, for example, CURCUWIN® (available from, e.g., OmniActive Health Technologies), MERIVA® (Indena S.p.A.; Milan, Italy), and CURCUGEN® (Dolcas Biotech, LLC; Landing, NJ).

A composition provided herein can contain one or more than one bioavailability-enhanced curcuminoids. A composition provided herein can contain any appropriate amount of a bioavailability-enhanced curcuminoid. In some cases, a dietary supplement can contain from about 1 wt % to about 20 wt % (e.g., from about 1 wt % to about 15 wt %, from about 1 wt % to about 10 wt %, from about 1 wt % to about 8 wt %, from about 1 wt % to about 6 wt %, from about 2 wt % to about 20 wt %, from about 2 wt % to about 15 wt %, from about 2 wt % to about 10 wt %, from about 2 wt % to about 8 wt %, from about 2 wt % to about 6 wt %, from about 4 wt % to about 20 wt %, from about 4 wt % to about 15 wt %, from about 4 wt % to about 10 wt %, from about 4 wt % to about 8 wt %, from about 4 wt % to about 6 wt %, from about 6 wt % to about 20 wt %, from about 6 wt % to about 15 wt %, from about 6 wt % to about 10 wt %, from about 6 wt % to about 8 wt %, from about 8 wt % to about 20 wt %, from about 8 wt % to about 15 wt %, from about 8 wt % to about 10 wt %, from about 10 wt % to about 20 wt %, from about 10 wt % to about 15 wt %, from about 15 wt % to about 20 wt %, about 2 wt %, about 4 wt %, about 6 wt %, about 8 wt %, about 10 wt %, about 15 wt %, or about 20 wt %)) of a bioavailability-enhanced curcuminoid. For example, a composition can be formulated to contain about 6% of a bioavailability-enhanced curcuminoid. In some cases, a dietary supplement can contain from about 1 mg to about 500 mg (e.g., from about 5 mg to about 450 mg, from about 10 mg to about 400 mg, from about 15 mg to about 350 mg, from about 20 mg to about 300 mg, from about 25 mg to about 250 mg, from about 30 mg to about 200 mg, from about 35 mg to about 150 mg, from about 35 mg to about 100 mg, from about 40 mg to about 75 mg, from about 40 mg to about 60 mg, about 10 mg, about 20 mg, about 30 mg, about 40 mg, about 50 mg, about 60 mg, about 70 mg, about 80 mg, about 90 mg, or about 100 mg) of a bioavailability-enhanced curcuminoid. For example, a composition can be formulated to contain 50 mg of a bioavailability-enhanced curcuminoid. In some cases, a composition provided herein can be formulated to contain an amount of a bioavailability-enhanced curcuminoid such that a serving (a single dose) of from about 1 mg to about 500 mg (e.g., from about 5 mg to about 450 mg, from about 10 mg to about 400 mg, from about 15 mg to about 350 mg, from about 20 mg to about 300 mg, from about 25 mg to about 250 mg, from about 30 mg to about 200 mg, from about 35 mg to about 150 mg, from about 35 mg to about 100 mg, from about 40 mg to about 75 mg, from about 40 mg to about 60 mg, about 10 mg, about 20 mg, about 30 mg, about 35 mg, about 40 mg, about 45 mg, about 50 mg, about 55 mg, about 60 mg, about 65 mg, about 70 mg, about 80 mg, about 90 mg, or about 100 mg) of the curcuminoid can be effectively delivered to a mammal (e.g., a human).

In some cases, the bioavailability-enhanced curcuminoid can be a component of a turmeric extract. In some cases, the turmeric extract can be obtained using standard extraction techniques. In some cases, a composition provided herein can contain any appropriate amount of a turmeric extract. For example, a dietary supplement can contain from about 5 wt % to about 50 wt % (e.g., from about 5 wt % to about 50 wt %, from about 5 wt % to about 40 wt %, from about 5 wt % to about 30 wt %, from about 5 wt % to about 20 wt %, from about 10 wt % to about 40 wt %, from about 10 wt % to about 30 wt %, from about 10 wt % to about 20 wt %, from about 20 wt % to about 50 wt %, from about 20 wt % to about 40 wt %, from about 20 wt % to about 30 wt %, from about 30 wt % to about 50 wt %, from about 30 wt % to about 40 wt %, from about 40 wt % to about 50 wt %, about 10 wt %, about 20 wt %, about 30 wt %, about 40 wt %, or about 50 wt %) of the turmeric extract. For example, a composition can be formulated to contain about 30 wt % of turmeric extract. In some cases, a dietary supplement can contain from about 1 mg to about 2500 mg (e.g., from about 10 mg to about 1000 mg, from about 25 mg to about 750 mg, from about 50 mg to about 500 mg, from about 100 mg to about 500 mg, from about 200 mg to about 300 mg, about 100 mg, about 150 mg, about 200 mg, about 250 mg, about 300 mg, about 350 mg, or about 400 mg) of turmeric extract. In some cases, a composition can be formulated to contain an amount of the turmeric extract such that a serving (e.g., a single dose) of about 1 mg to about 2500 mg (e.g., from about 10 mg to about 1000 mg, from about 25 mg to about 750 mg, from about 50 mg to about 500 mg, from about 100 mg to about 500 mg, from about 200 mg to about 300 mg, about 100 mg, about 150 mg, about 200 mg, about 250 mg, about 300 mg, about 350 mg, or about 400 mg) of the turmeric extract can be conveniently administered. For example, a composition can be formulated to contain 250 mg of a turmeric extract containing a bioavailability-enhanced curcuminoid.

### Iridoid

The compositions provided herein can contain one or more iridoids (e.g., iridoid glycosides). Examples of iridoids include, without limitation, harpagoside, loganin, sweroside, vogeloside, and epi-vogeloside. In some cases, an iridoid can be a component of a plant extract. For example, an iridoid can be a component of an extract of Devil's Claw. A Devil's Claw extract can be made using, for example, ethanol or hydroalcoholic extraction of a Devil's Claw plant. In some cases, iridoids and/or plant extracts containing iridoids (e.g., Devil's Claw) can be obtained commercially. For example, Devil's Claw extract can be obtained from Pharmline Inc. (NY, USA) or from Givaudan (Vernier, Switzerland).

A composition provided herein can contain one or more than one iridoid (e.g., iridoid glycoside). A composition can contain any appropriate amount of an iridoid. In some cases, a dietary supplement can contain from about 0.005 wt % to about 2 wt % (e.g., from about 0.005 wt % to about 1.6 wt %, from about 0.005 wt % to about 1.2 wt %, from about 0.005 wt % to about 0.8 wt %, from about 0.005 wt % to about 0.4 wt %, from about 0.005 wt % to about 0.2 wt %, from about 0.01 wt % to about 2 wt %, from about 0.01 wt % to about 1.6 wt %, from about 0.01 wt % to about 1.2 wt %, from about 0.01 wt % to about 0.8 wt %, from about 0.01 wt % to about 0.4 wt %, from about 0.01 wt % to about 0.2 wt %, from about 0.05 wt % to about 2 wt %, from about 0.05 wt % to about 1.6 wt %, from about 0.05 wt % to about 1.2 wt %, from about 0.05 wt % to about 0.8 wt %, from about 0.05 wt % to about 0.4 wt %, from about 0.05 wt % to about 0.2 wt %, from about 0.1 wt % to about 2 wt %, from about 0.1 wt % to about 1.6 wt %, from about 0.1 wt % to about 1.2 wt %, from about 0.1 wt % to about 0.8 wt %, from about 0.1 wt % to about 0.4 wt %, from about 0.1 wt % to about 0.2 wt %, about 0.01 wt %, about 0.1 wt %, about 0.15 wt %, about 0.16 wt %, about 0.2 wt %, about 0.4 wt %, about 0.8 wt %, about 1.2 wt %, about 1.6 wt %, or about 2 wt %) of an iridoid. For example, a composition can contain 0.16% of an iridoid (e.g., an iridoid glycoside, such as harpagoside). In some cases, a dietary supplement can contain from about 0.1 mg to about 60 mg (e.g., from about 0.1 mg to about 40 mg, from about 0.1 mg to about 20 mg, from about 0.1 mg to about 10 mg, from about 0.1 mg to about 5 mg, from about 0.1 mg to about 2 mg, from about 1 mg to about 60 mg, from about 1 mg to about 40 mg, from about 1 mg to about 20 mg, from about 1 mg to about 10 mg, from about 1 mg to about 5 mg, from about 1 mg to about 2 mg, from about 2 mg to about 60 mg, from about 2 mg to about 40 mg, from about 2 mg to about 20 mg, from about 2 mg to about 10 mg, from about 2 mg to about 5 mg, from about 5 mg to about 60 mg, from about 5 mg to about 40 mg, from about 5 mg to about 20 mg, from about 5 mg to about 10 mg, from about 10 mg to about 60 mg, from about 10 mg to about 40 mg, from about 10 mg to about 20 mg, from about 20 mg to about 60 mg, from about 20 mg to about 40 mg, from about 40 mg to about 60 mg, about 1 mg, about 1.3 mg, about 1.5 mg, about 2 mg, or about 2.5 mg) of an iridoid. In some cases, a composition can be formulated to contain an amount of iridoid such that a serving (e.g., a single dose) of about 0.5 mg to about 10 mg (e.g., from about 0.5 mg to about 5 mg, from about 0.5 mg to about 4 mg, from about 0.5 mg to about 3 mg, from about 0.5 mg to about 2 mg, from about 1 mg to about 5 mg, from about 1 mg to about 4 mg, from about 1 mg to about 3 mg, from about 1 mg to about 2 mg, from about 2 mg to about 5 mg, from about 2 mg to about 4 mg, from about 3 mg to about 5 mg, about 1 mg, about 1.3 mg, about 1.5 mg, about 2 mg, or about 2.5 mg) of the iridoid can be conveniently administered. For example, a composition can be formulated to contain about 1.3 mg of an iridoid.

In some cases, an iridoid can be a component of a plant extract. For example, an iridoid can be a component of a Devil's Claw extract. In some cases, a Devil's Claw extract can be obtained using standard extraction techniques. A composition can contain any appropriate amount of a plant extract, such as a Devil's claw extract. For example, a dietary supplement can contain from about 1 wt % to about 20 wt % (e.g., from about 1 wt % to about 15 wt %, from about 1 wt % to about 10 wt %, from about 2 wt % to about 20 wt %, from about 2 wt % to about 10 wt %, from about 4 wt % to about 10 wt %, from about 4 wt % to about 9 wt %, from about 6 wt % to about 10 wt %, from about 6 wt % to about 9 wt %, from about 8 wt % to about 10 wt %, about 2 wt %, about 4 wt %, about 6 wt %, about 8 wt %, about 8.5 wt %, about 8.6 wt. %, about 8.7 wt %, or about 10 wt %) of the plant extract. For example, a composition can contain about 8.7 wt % of a Devil's claw extract. In some cases, a dietary supplement can contain from about 1 mg to about 500 mg (e.g., from about 5 mg to about 450 mg, from about 10 mg to about 400 mg, from about 15 mg to about 350 mg, from about 20 mg to about 300 mg, from about 25 mg to about 250 mg, from about 30 mg to about 200 mg, from about 35 mg to about 150 mg, from about 35 mg to about 100 mg, from about 40 mg to about 75 mg, from about 40 mg to about 60 mg, about 30 mg, about 35 mg, about 40 mg, about 45 mg, about 50 mg, about 55 mg, about 60 mg, about 65 mg, of about 70 mg) of a Devil's claw extract. In some cases, a composition can be formulated to contain an amount of Devil's claw extract such that a serving (e.g., a single dose) of from about 1 mg to about 500 mg (e.g., from about 5 mg to about 450 mg, from about 10 mg to about 400 mg, from about 15 mg to about 350 mg, from about 20 mg to about 300 mg, from about 25 mg to about 250 mg, from about 30 mg to about 200 mg, from about 35 mg to about 150 mg, from about 35 mg to about 100 mg, from about 40 mg to about 75 mg, from about 40 mg to about 60 mg, about 30 mg, about 35 mg, about 40 mg, about 45 mg, about 50 mg, about 55 mg, about 60 mg, about 65 mg, of about 70 mg) of the Devil's claw extract can be conveniently administered. For example, a composition can be formulated to contain about 52.5 mg of a Devil's Claw extract.

### Green Tea Extract

The compositions provided herein can contain one or more green tea extracts. In some cases, a green tea extract can be an extract derived from Camellia sinensis. Any method can be used to obtain a green tea extract. For example, a green tea extract can be obtained by drying (e.g., freeze drying or spray drying) a liquor from an alcoholic, hydroalcoholic, or other hydrocarbon extraction of green tea leaves. In some cases, a green tea extract can be dried and standardized to contain at least about 25 percent total phenols. A green tea extract can contain catechin, epicatechin, gallocatechin, epigallocatechin, epicatechin gallate, epicatechin gallate, and epigallocatechin gallate. Typically, a composition provided herein contains a green tea extract having at least about 25 percent of catechin group compounds. A green tea extract can be caffeinated or decaffeinated. In some cases, a green tea extract can be obtained commercially. For example, a green tea extract can be obtained from Buckton Scott Nutrition, Inc. (Fairfield, NJ), Pure World, Inc. (Hackensack, NJ), Sabinsa Corporation (Piscataway, NJ), or Stauber Performance Ingredients Inc., (Fullerton, CA).

A composition provided herein can contain one or more than one green tea extract. In some cases, a composition can contain any amount of a green tea extract. For example, about 5 wt % to about 50 wt % (e.g., from about 5 wt % to about 50 wt %, from about 5 wt % to about 40 wt %, from about 5 wt % to about 30 wt %, from about 5 wt % to about 20 wt %, from about 10 wt % to about 40 wt %, from about 10 wt % to about 30 wt %, from about 10 wt % to about 20 wt %, from about 20 wt % to about 50 wt %, from about 20 wt % to about 40 wt %, from about 20 wt % to about 30 wt %, from about 30 wt % to about 50 wt %, from about 30 wt % to about 40 wt %, from about 40 wt % to about 50 wt %, about 10 wt %, about 20 wt %, about 30 wt %, about 40 wt %, or about 50 wt %) of a dietary supplement can be a green tea extract. In some embodiments, a composition can be formulated to contain about 40 wt % of a green tea extract. The dietary supplement compositions provided herein can contain from about 1 mg to about 2500 mg (e.g., from about 10 mg to about 1000 mg, from about 25 mg to about 750 mg, from about 50 mg to about 500 mg, from about 100 mg to about 500 mg, from about 200 mg to about 300 mg, about 100 mg, about 150 mg, about 200 mg, about 250 mg, about 300 mg, about 350 mg, or about 400 mg) of a green tea extract. In some cases, a composition can be formulated to contain an amount of a green tea extract such that a serving (e.g., a single dose) of about 1 mg to about 2500 mg (e.g., from about 10 mg to about 1000 mg, from about 25 mg to about 750 mg, from about 50 mg to about 500 mg, from about 100 mg to about 500 mg, from about 200 mg to about 300 mg, about 100 mg, about 150 mg, about 200 mg, about 250 mg, about 300 mg, about 350 mg, or about 400 mg) of a green tea extract can be conveniently administered. For example, a composition can be formulated to contain about 250 mg of a green tea extract.

### Ginger

The compositions provided herein can contain one or more ginger components. Examples of ginger components include, without limitation, dried ginger (e.g., dried gingerroot), ginger oil, and ginger extracts. In some cases, a ginger component can be a ginger root extract. A ginger component can be obtained from any of the estimated 1300 species of plants that belong to the Zingiberaceae family. For example, a ginger component can be derived from Zingiber officinale, Alpinia officnarum, or Alpinia galanga.

Any method can be used to prepare a ginger component. For example, standard harvesting and drying methods can be used to prepare dried gingerroot. In some cases, ginger oil can be obtained (e.g., using ethanol or hydroalcoholic extraction) and processed with cellulose for making tablet, powder, or capsule compositions. Such extracts can be standardized to, for example, a composition of about 5% to about 75% (from about 10% to about 65%, from about 20% to about 55%, from about 30% to about 45%, from about 40% to about 45%) gingerol or shogaol. In some cases, ginger extracts can be standardized to about 5% gingerols or shogaol. In some cases, ginger components can be obtained commercially. For example, dried ginger, ginger oil, and ginger extract can be obtained from BattleChem Inc., (CA, USA) or from Givaudan.

A composition provided herein can contain one or more than one ginger component. For example, a dietary supplement can contain dried gingerroot as well as a ginger root extract. A composition can contain any appropriate amount of a ginger component. For example, about 1 wt % to about 20 wt % (e.g., from about 1 wt % to about 15 wt %, from about 1 wt % to about 10 wt %, from about 2 wt % to about 20 wt %, from about 2 wt % to about 10 wt %, from about 4 wt % to about 10 wt %, from about 4 wt % to about 9 wt %, from about 6 wt % to about 10 wt %, from about 6 wt % to about 9 wt %, from about 8 wt % to about 10 wt %, about 2 wt %, about 4 wt %, about 6 wt %, about 8 wt %, about 8.5 wt %, about 8.6 wt. %, about 8.7 wt %, or about 10 wt %) of a dietary supplement can be a ginger component. In some cases, a dietary supplement can contain from about 1 mg to about 500 mg (e.g., from about 5 mg to about 450 mg, from about 10 mg to about 400 mg, from about 15 mg to about 350 mg, from about 20 mg to about 300 mg, from about 25 mg to about 250 mg, from about 30 mg to about 200 mg, from about 35 mg to about 150 mg, from about 35 mg to about 100 mg, from about 40 mg to about 75 mg, from about 40 mg to about 60 mg, about 35 mg, about 40 mg, about 45 mg, about 50 mg, about 55 mg, about 60 mg, or about 65 mg) of a ginger component. A composition can be formulated to contain an amount of a ginger component such that a single dose of from about 5 mg to about 350 mg (e.g., from about 50 mg to about 300, from about 50 mg to about 250, from about 50 mg to about 200, from about 50 mg to about 150, from about 50 mg to about 100, from about 300 mg to about 250, from about 300 mg to about 200, from about 300 mg to about 150, from about 300 mg to about 100, from about 250 mg to about 200, from about 250 mg to about 150, from about 250 mg to about 100, from about 200 mg to about 150, from about 200 mg to about 100, or from about 150 mg to about 100, about 35 mg, about 40 mg, about 45 mg, about 50 mg, about 55 mg, about 60 mg, or about 65 mg) of the ginger component can be conveniently administered. For example, a composition can be formulated to contain about 52.5 mg of a ginger component. When ginger root extract is used, a composition can be formulated to contain an amount of ginger root extract such that a daily dose of from about 50 mg to about 1000 mg (e.g., between 50 mg and 900 mg between 50 mg and 850 mg, between 50 mg and 750 mg, between 100 mg and 1000 mg, between 200 mg and 1000 mg, between 350 mg and 1000 mg, between 450 mg and 1000 mg, between 600 mg and 1000 mg, between 700 mg and 1000 mg, between 300 mg to 900 mg, and between 600 mg and 800 mg ginger root extract) of the ginger root extract can be conveniently administered.

### Dietary Supplement Formulations

Provided herein are compositions (e.g., dietary supplements) containing a combination of a bioavailability-enhanced curcuminoid, an iridoid, a green tea extract, and a ginger component. In some cases, the composition can contain a combination of a bioavailability-enhanced curcuminoid, a Devil's Claw extract, a green tea extract, and a ginger component. The compositions provided herein can be used to relieve pain, inflammation, and discomfort associated with conditions such as, for example, OA, diabetes, heart disease, and depression. In some cases, this document provides methods for promoting a healthy inflammatory response to normal daily activities (e.g., physical activity) in a mammal (e.g., a human), promoting a healthy inflammatory response to exercise induced inflammation, relieving or reducing pain, inflammation, and/or discomfort due to, for example, diseases associated with inflammation, such as arthritis, OA, diabetes, heart disease, and depression. The methods provided herein include administering, to a mammal, a composition provided herein.

The compositions provided herein are intended to be ingested (e.g., orally or intragastrically), but they also can be administered to a mammal by other routes. For example, a composition provided herein can be administered nasally, intravenously, intramuscularly, subcutaneously, sublingually, intrathecally, transdermally, or intradermally. The route of administration can depend on a variety of factors, such as the environment (e.g., the circumstances resulting in the condition or symptoms) and therapeutic goals.

When administered orally, a composition provided herein can be in the form of a tablet, capsule, or powder. In some cases, tablets and powders can be configured to have a unit dosage equal to a daily desired dosage. For example, if a desired daily dosage is 1000 mg of a particular composition, each tablet can be 1000 mg in weight. As used herein, the term “mammals” generally refers to humans, but the term also can include domesticated mammals (e.g., dogs, cats, and livestock such as cows, horses, pigs, or sheep) and research animals (e.g., mice, rats, and rabbits) in which reducing pain, inflammation, and/or stiffness is desirable.

The dosage of a particular composition will depend on many factors, including the mode of administration. In some cases, a dietary supplement provided herein can be formulated in a dose such that an individual receives about 50 mg curcuminoids (e.g., via about 250 mg of turmeric extract), about 52.5 mg of Devil's Claw extract, about 52.5 mg of ginger root extract, and about 250 mg of green tea extract, blended in a single tablet or capsule. In some cases, a dietary supplement provided herein can be formulated in a dose such that an individual receives about 50 mg curcuminoids (e.g., via about 250 mg of turmeric extract), about 52.5 mg of Devil's Claw extract, about 52.5 mg of ginger root extract, and about 250 mg of green tea extract, provided in two or more (e.g., two, three, four, or five) tablets or capsules.

By way of example, a composition provided herein can be in the form of a liquid, solution, suspension, tablet, powder, cream, mist, atomized vapor, aerosol, soft gelatin capsules, or hard gelatin capsules. Commercial dietary supplements are generally formulated for oral administration. For oral administration, tablets or capsules can be prepared by conventional means with pharmaceutically acceptable excipients such as binding agents, fillers, lubricants, disintegrants, or wetting agents. The tablets can be coated by methods known in the art. Liquid preparations for oral administration can take the form of, for example, solutions, syrups, or suspension, or they can be presented as a dry product for constitution with saline or other suitable liquid vehicle before use. Liquid preparations also can contain pharmaceutically acceptable additives such as suspending agents, emulsifying agents, non-aqueous vehicles, preservatives, buffer salts, flavoring agents, coloring agents, and sweetening agents as appropriate. Preparations for oral administration can be suitably formulated to give controlled release of the compound. Typically, the compositions provided herein are in a powder or tablet form with a fast disintegration time.

In some cases, a composition provided herein can contain a pharmaceutically acceptable carrier for in vivo administration to a mammal. Suitable pharmaceutically acceptable carriers include, without limitation, sterile aqueous or non-aqueous solutions, suspensions, and emulsions. Non-limiting examples of non-aqueous solvents include, without limitation, propylene glycol, polyethylene glycol, vegetable oils, and injectable organic esters. Aqueous carriers include, without limitation, water, alcohol, saline, and buffered solutions. Pharmaceutically acceptable carriers also can include physiologically acceptable aqueous vehicles (e.g., physiological saline) or other known carriers appropriate to specific routes of administration. Preservatives, flavorings, and other additives such as, for example, proteins, anti-microbials, chelating agents, inert gases, and the like also can be present in a composition provided herein.

The compositions provided herein can be formulated for administration to a mammal at any appropriate frequency and for any appropriate duration. For example, a composition provided herein can be formulated for ingestion by a mammal at any frequency that reduces inflammation and/or pain in the mammal, without producing significant toxicity to the mammal. For example, the frequency of administration (e.g., frequency of administration of a single serving or dose) of a composition provided herein can be from about one to four times daily to about once a week (e.g., once daily, twice daily, three times daily, four times daily, one to two times daily, two to three times daily, three to four times daily, about every other day, about three times a week, about twice a week, or about once a week). The frequency of administration of a composition provided herein can remain constant or can be variable. Various factors can influence the actual frequency of administration used for a particular application. For example, the effective amount, duration of treatment, use of multiple anti-inflammatory agents, route of administration, and severity of the mammal's condition may require an increase or decrease in administration frequency.

An effective duration for administering a composition provided herein can be any duration that reduces a symptom of cancer in the mammal, reduces the number of cancer cells in the mammal, reduces inflammation and/or pain in the mammal, without producing significant toxicity to the mammal. In some cases, the effective duration can vary from several days to several months, to several years, or longer. Multiple factors can influence the actual effective duration used for a particular treatment. For example, an effective duration can vary with the effective amount, frequency of administration, use of multiple anti-inflammatory agents, route of administration, and severity of the mammal's condition.

### EXEMPLARY EMBODIMENTS

Embodiment 1 is a dietary supplement comprising: one or more bioavailability-enhanced curcuminoids, one or more iridoids, green tea extract, and one or more ginger components.

Embodiment 2 is the dietary supplement of embodiment 1, wherein the one or more bioavailability-enhanced curcuminoids include a component of a turmeric extract.

Embodiment 3 is the dietary supplement of embodiment 1 or embodiment 2, wherein the one or more iridoids are selected from the group consisting of: harpagoside, loganin, sweroside, vogeloside, and epi-vogeloside.

Embodiment 4 is the dietary supplement of embodiment 3, wherein the one or more iridoid include a component of a plant extract.

Embodiment 5 is the dietary supplement of embodiment 4, wherein the plant extract is a Devil's Claw extract.

Embodiment 6 is the dietary supplement of any one of embodiments 1 to 5, wherein the green tea extract comprises one or more of: catechin, epicatechin, gallocatechin, epigallocatechin, epicatechin gallate, epicatechin gallate, and epigallocatechin gallate.

Embodiment 7 is the dietary supplement of any one of embodiments 1 to 5, wherein the green tea extract comprises epigallocatechin gallate.

Embodiment 8 is the dietary supplement of any one of embodiments 1 to 7, wherein one or more ginger components include a ginger root extract.

Embodiment 9 is the dietary supplement of any one of embodiments 1 to 8, wherein the dietary supplement is in the form of a tablet, a capsule, a powder, or a liquid.

Embodiment 10 is a dietary supplement comprising: (a) from about 1 to about 150 mg of bioavailability-enhanced curcuminoid, (b) from about 40 to about 60 mg of Devil's Claw extract, (c) from about 150 to about 350 mg of green tea extract, and (d) from about 40 to about 60 mg of ginger component.

Embodiment 11 is the dietary supplement of embodiment 10, wherein the dietary supplement comprises: (a) about 50 mg/serving of the bioavailability-enhanced curcuminoid, (b) about 52.5 mg/serving of the Devil's Claw extract, (c) about 250 mg/serving of the green tea extract, and (d) about 52.5 mg/serving of the ginger component.

Embodiment 12 is a dietary supplement consisting essentially of: (a) from about 1 to about 150 mg of bioavailability-enhanced curcuminoid, (b) from about 40 to about 60 mg of Devil's Claw extract, (c) from about 150 to about 350 mg of green tea extract, and (d) from about 40 to about 60 mg of ginger component.

Embodiment 13 is the dietary supplement of embodiment 12, wherein the dietary supplement consists essentially of: (a) about 50 mg/serving of the bioavailability-enhanced curcuminoid, (b) about 52.5 mg/serving of the Devil's Claw extract, (c) about 250 mg/serving of the green tea extract, and (d) about 52.5 mg/serving of the ginger component.

Embodiment 14 is a method for promoting a healthy inflammatory response to normal daily activity in a mammal, wherein the method comprises administering to the mammal the dietary supplement of any one of embodiments 1 to 13.

Embodiment 15 is the method of embodiment 14, comprising administering the dietary supplement to the mammal on a daily basis for at least one week.

Embodiment 16 is the method of embodiment 14 or embodiment 15, wherein the administering is effective to reduce inflammation in the mammal.

Embodiment 17 is the method of any one of embodiments 14 to 16, wherein the mammal is a human.

Embodiment 18 is a method for promoting a healthy inflammatory response to exercise-induced inflammation in a mammal, wherein the method comprises administering to the mammal the dietary supplement of any one of embodiments 1 to 13.

Embodiment 19 is the method of embodiment 18, comprising administering the dietary supplement to the mammal on a daily basis for at least one week.

Embodiment 20 is the method of embodiment 18 or embodiment 19, wherein the administering is effective to reduce inflammation in the mammal.

Embodiment 21 is the method of any one of embodiments 18 to 20, wherein the mammal is a human.

The invention will be further described in the following example, which does not limit the scope of the invention described in the claims.

### EXAMPLE

Different curcuminoid sources (turmeric extract, CURCUWIN®, MERIVA®, CURCUGEN®, and CU+) had different degrees of bioavailability, with CU+ having superior bioavailability compared to the other enhanced curcuminoids that were tested (TABLE 1). The potent anti-inflammatory effects of CU+ were more balanced with a corresponding dose of green tea and smaller quantities of other anti-inflammatory mechanisms for more efficient management of symptoms.

In a randomized, single-blind, within-subjects design, participants completed a crossover study to determine the effects of the Test Formula versus an existing formulation (Benchmark) that did not contain curcuminoid that was bioavailability enhanced. On day 2, separated by a washout period of at least 3 days, participants reported to the laboratory after an overnight fast. Baseline blood samples were taken, and participants consumed 1 serving of either the Test Formula containing CU+ or the Benchmark formulation, followed by extraction of blood samples again after 2 hours. Reactive oxygen species (ROS) was measured in the collected capillary blood samples using the vitascreen (EPR; Noxygen) protocol with and without the addition of 40 ng/mL TNF-α at a 1:1 ratio with whole blood and spin probe. TNF-α was added to observe the efficacy of the formulas to resist high-inflammation stress conditions.

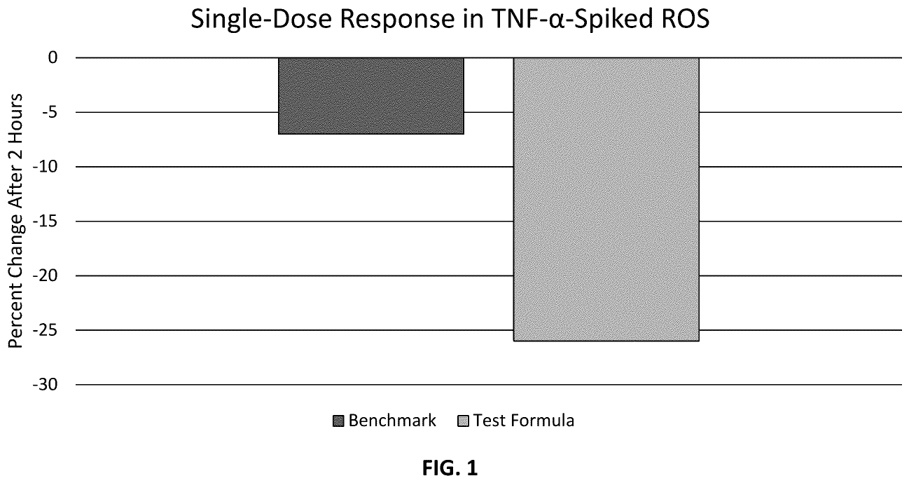

Samples from subjects who received the Test Formula showed a 26% reduction in ROS levels (a marker of oxidative stress) compared to the samples from subjects who received the Benchmark formulation, which showed a 7% reduction in ROS (FIG. 1). The Test Formula also resulted in over 3 times more efficacious inflammation resistance compared to the Benchmark formula when expressed as a percent change.

TABLE 1
Bioavailability of enhanced curcuminoids

| | | AUC | Relative | | Equivalent |
|---|---|---|---|---|---|
| | Dose (mg | (0-12 h) | Absorption | Number | grams of |
| Material | curcuminoids) | (ng/ml*h) | (0-12 h) | of Pills | turmeric |
| Turmeric extract | 1800 | 10.8 | 1.0 | 4 | 60 |
| (95% curcuminoids) | | | | | |
| CURCUWIN ® | 376 | 307.6 | 136.3 | 1 | 8.178 |
| MERIVA ® | 376 | 28.7 | 12.7 | 1 | 762 |
| CURCUGEN ® | 376 | 5.8 | 2.6 | 1 | 156 |
| CU+ (observed) | 50 | 182.6 | 98.78 | 1 | 5.927 |
| CU+ (observed) | 100 | 419.8 | 113.33 | 1 | 6.800 |
| CU+ (calculated) | 376 | 1578.4* | 426.12* | 1 | 25.567 |
| CU+ (calculated) | 376 | 1729.1** | 193.65** | 1 | 11.619 |

*The dependent variables of the CU + 100 mg treatment data were multiplied by 3.76 to arrive at a number for comparison to other branded material on an equal dosage basis.
**As an alternate calculation, the difference between the dependent variables for the 50 mg and 100 mg CU + doses studied were used to represent the increase for every 50 mg increase in CU + administered and added to the relative absorption of 100 mg CU+ (e.g., relative absorption: 113.33 − 98.78 = 14.55; 376 − 100 = 276, 276/50 = 5.52, 5.52 × 14.55 = 80.32, 80.32 + 113.33 = 193.65).

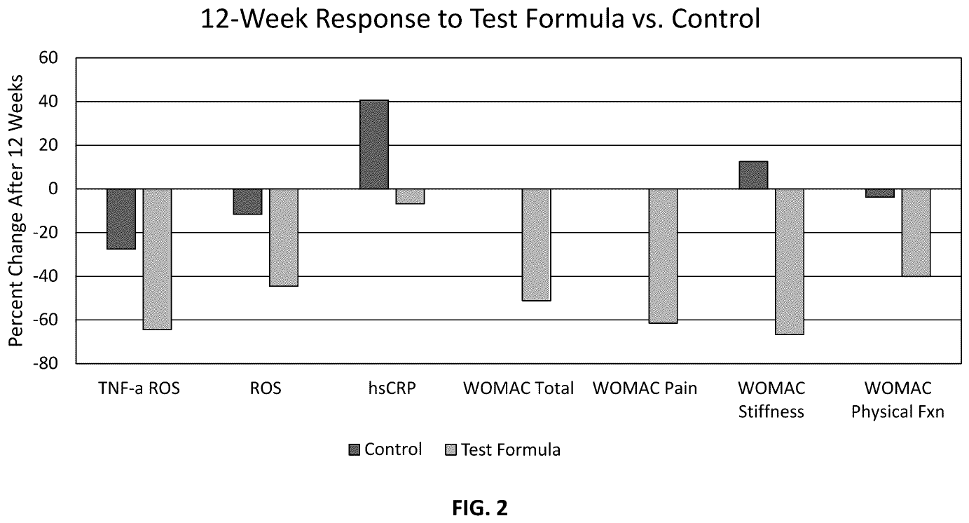

To test the anti-inflammatory effects of the Test Formula in in vivo, a 12-week randomized, single-blind, parallel arm experiment was designed. After 12 weeks, the levels of hsCRP and Tumor necrosis factor-alpha (TNF-α) were measured in peripheral blood samples. ROS levels with and without the addition of TNF-α were measured in capillary blood using electron paramagnetic resonance (EPR). As determined by the WOMAC Index, the Test Formula showed greater improvement in joint comfort compared to the control (FIG. 2).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A dietary supplement comprising:
a bioavailability-enhanced curcuminoid,
an iridoid,
a green tea extract, and
a ginger component.

2. The dietary supplement of claim 1, wherein said bioavailability-enhanced curcuminoid is a component of a turmeric extract.

3. The dietary supplement of claim 1, wherein said iridoid is selected from the group consisting of: harpagoside, loganin, sweroside, vogeloside, and epi-vogeloside.

4. The dietary supplement of claim 3, wherein said iridoid is a component of a plant extract.

5. The dietary supplement of claim 4, wherein said plant extract is a Devil's Claw extract.

6. The dietary supplement of claim 1, wherein said green tea extract comprises one or more of: catechin, epicatechin, gallocatechin, epigallocatechin, epicatechin gallate, epicatechin gallate, and epigallocatechin gallate.

7. The dietary supplement of claim 1, wherein said green tea extract comprises epigallocatechin gallate.

8. The dietary supplement of claim 1, wherein said ginger component is a ginger root extract.

9. The dietary supplement of claim 1, wherein said dietary supplement is in the form of a tablet, a capsule, a powder, or a liquid.

10. A dietary supplement comprising:
(a) from about 1 to about 150 mg of bioavailability-enhanced curcuminoid,
(b) from about 40 to about 60 mg of Devil's Claw extract,
(c) from about 150 to about 350 mg of green tea extract, and
(d) from about 40 to about 60 mg of ginger component.

11. The dietary supplement of claim 10, wherein said dietary supplement comprises:
(a) about 50 mg/serving of said bioavailability-enhanced curcuminoid,
(b) about 52.5 mg/serving of said Devil's Claw extract,
(c) about 250 mg/serving of said green tea extract, and
(d) about 52.5 mg/serving of said ginger component.

12. A dietary supplement consisting essentially of:
(a) from about 1 to about 150 mg of bioavailability-enhanced curcuminoid,
(b) from about 40 to about 60 mg of Devil's Claw extract,
(c) from about 150 to about 350 mg of green tea extract, and
(d) from about 40 to about 60 mg of ginger component.

13. The dietary supplement of claim 12, wherein said dietary supplement consists essentially of:
(a) about 50 mg/serving of said bioavailability-enhanced curcuminoid,
(b) about 52.5 mg/serving of said Devil's Claw extract,
(c) about 250 mg/serving of said green tea extract, and
(d) about 52.5 mg/serving of said ginger component.

14. A method for promoting a healthy inflammatory response to normal daily activity in a mammal, wherein said method comprises administering to said mammal the dietary supplement of claim 1.

15. The method of claim 14, comprising administering said dietary supplement to said mammal on a daily basis for at least one week.

16. The method of claim 14, wherein said administering is effective to reduce inflammation in said mammal.

17. The method of claim 14, wherein said mammal is a human.

18. A method for promoting a healthy inflammatory response to exercise-induced inflammation in a mammal, wherein said method comprises administering to said mammal the dietary supplement of claim 1.

19. The method of claim 18, comprising administering said dietary supplement to said mammal on a daily basis for at least one week.

20. The method of claim 18, wherein said administering is effective to reduce inflammation in said mammal.
